(19) United States
(12) Patent Application Publication
(10) Pub. No.: US 2012/0149872 A1
(43) Pub. Date: Jun. 14, 2012

(54) Channel-based purification device

(76) Inventor: Phillip Belgrader, Severna Park, MD (US)

(21) Appl. No.: 12/003,416

(22) Filed: Dec. 26, 2007

Related U.S. Application Data
(60) Provisional application No. 60/877,353, filed on Dec. 28, 2006.

(51) Int. Cl. C07K 1/14; B01D 17/00; C07H 1/06

(52) U.S. Cl. 530/344; 536/25.4; 422/285

(57) ABSTRACT

The present invention relates to a device for purifying an analyte from a fluid sample. The device comprises a channel or tubing having an inner surface that binds to the analyte of interest in the fluid sample. As the fluid sample flows through the channel, the analyte of interest binds to the inner wall of the channel. The bound analyte is then eluted using a small bolus of elution buffer. The channel generates a high surface area for capturing the analyte in a large volume sample, but allows low liquid elution volume for concentrating the analyte into a small volume.

### CROSS-REFERENCE TO RELATED APPLICATION(S)

This application claims priority from U.S. Provisional Application No. 60/877,353, filed Dec. 28, 2006 and entitled “CHANNEL-BASED PURIFICATION DEVICE,” the content of which is incorporated herein in its entirety to the extent that it is consistent with this invention and application.

### TECHNICAL FIELD

The present invention relates generally to purification devices. Specifically, the present invention relates to a channel-based device for purifying an analyte in a fluid sample.

### BACKGROUND OF THE INVENTION

Qiagen kits, the most practiced commercial method for nucleic acid purification, involve moving a volume of sample mixed with a chaotropic agent like guanidine through a high surface area glass membrane. Nucleic acids are induced to interact with the hydroxyl groups on the silica surface and are essentially extracted from the sample. Proteins remain fairly soluble in the guanidine solution, and any proteins that may co-precipitate with the nucleic acids on the silica membrane are washed from the surface using ethanol. Nucleic acids are eluted from the silica membrane using water or Tris buffer. Related approaches include silica gel, packed glass bead column, micropillar chip, and paramagnetic beads. Limitations to these approaches may include low concentration factor, expense, slow speed, highly variable recoveries, clogging, low binding capacity, open system, complex automation and packaging, and/or lack of reusability. All these approaches, except for paramagnetic beads, involve creating a high surface area in a small space to extract and elute the nucleic acids. Paramagnetic beads (typically glass beads with an iron oxide core) are not constrained to some of the limitations created by the other methods, but controlling and packaging these beads into simple, repeatable devices is not trivial due to the nature of beads sticking to surfaces and getting trapped in pumps and valves. Therefore, robust automated protocols for paramagnetic beads have been limited to open, robotic pipetting stations.

Other approaches for sample preparation include the use of filters and frits. The GeneXpert® system (Cepheid, Sunnyvale, Calif.) is an example of a successful application of simple filter technology to process and PCR analyze air samples collected at Unite States Postal Service (USPS) mail sorting facilities. In this procedure, a fluidic cartridge, containing a porous filter, traps spores and any other large particles from a 1-ml input sample. Small inhibitors pass through the filter, especially after a wash step. The filter resides in a lysis chamber, and the trapped particles are concentrated into a smaller volume. Spores and other cells are lysed by a sonication horn that impinges the chamber. The crude lysate is pushed out of the chamber and subjected to PCR analysis. Despite utilizing crude lysate instead of purified nucleic acids, this approach has been demonstrated to be highly effective for the USPS application to monitor for Bacillus anthracis spores. This simple filter approach, however, is limited to certain types of sample matrices and large microbes. Accordingly, there still exists a need for reliable, rapid, and inexpensive device for nucleic acid and protein purification.

### SUMMARY OF THE INVENTION

One aspect of the present invention relates to a method for purifying an analyte from a fluid sample. The method comprises the steps of passing the fluid sample through a channel having an inner surface that binds to the analyte; and eluting bound analyte with a elution buffer.

Another aspect of the present invention relates to a device for purifying an analyte from a fluid sample. The device comprises a channel having an inner surface that binds to the analyte in the fluid sample; a fluid handling unit capable of injecting an elution buffer into the channel to elute analyte bound to the inner surface of the channel; and a heating unit capable of heating fluid in the channel for a desired period of time.

These and other embodiments of the invention are further described below with references to the following figures.

### BRIEF DESCRIPTION OF THE FIGURES

FIG. 1 is a schematic showing a fluidic solenoid (top, tubing wrapped around a heater), and a fluidic chip (bottom, channel or tubing inside a heating block).

FIG. 2 is a schematic showing a channel-based device adopted to several analytical platforms.

FIG. 3 is a schematic showing the working mechanism of a channel-based device for nucleic acid purification.

FIG. 4 is a schematic showing a channel-based approach to preparation of samples for PCR, immunoassays, and mass spectrometry.

FIG. 5 is a composite of pictures showing a microchannel nucleic acid concentrator (MNAC) testbed system.

FIG. 6A shows real-time PCR amplification of DNA samples purified and concentrated by silica tubing and guanidine-based bind buffer with a slow (15 min hold) elution protocol.

FIG. 6B shows real-time PCR amplification of DNA samples purified and concentrated by silica tubing and guanidine-based bind buffer with a fast (5 min hold) elution protocol.

FIG. 7 shows effective MNAC processing of a “dirty” air sample archive collected from a Biohazard detection system (BDS) installed at a USPS mail processing center. Top panel: real-time PCR analysis of input (unprocessed) and output (MNAC processed) samples. Bottom panel: real-time PCR analysis of diluted input and output samples to remove inhibition.

FIG. 8 shows examples of real-time PCR results from MNAC processing and concentration of (left) lysed Bacillus anthracis spores and (right) 1 ml M13 DNA input/5 μl output.

FIG. 9 shows the real-time PCR results on M13 DNA sample processing using a silicone tubing without guanidine and ethanol. DNA recovery was greater than 60% and concentration was almost 10-fold.

FIG. 10 shows the real-time PCR results of purification and concentration of M13 DNA in a dirty sample using silicone tubing and a modified elution buffer (1× PBS+0.01% SDS, pH 9.0) at 75° C.

FIGS. 11A and 11B show the real-time PCR (FIG. 11A) and real-time isothermal amplification (FIG. 11B) results of channel-based purification of DNA from lysed Bacillus anthracis spores. DNA in output sample is more concentrated (lower cycle threshold) than DNA in input sample.

FIGS. 12A and 12B show an aminated microfluidic serpentine channel in a PMMA chip (FIG. 12A, upper panel) and a syringe pump (FIG. 12A, lower panel) used for purification and concentration of DNA from lysed Bacillus subtilis spores, as well as the real-time PCR results of lysed Bacillus subtilis spores processed on the PMMA chip showing concentration of the Bacillus subtilis DNA (FIG. 12B).

FIGS. 13A and 13B show channel modules that comprise multiple channels and are capable of simultaneously processing multiple samples.

FIGS. 14A and 14B show an off-the-shelf glass serpentine channel device from Invenios (Santa Barbara, Calif.) (FIG. 14A) that is typically used for mixing two solutions, and real-time PCR results of input sample (10 ml) and concentrated sample (170 μl) (FIG. 14B).

FIG. 15 is a diagram showing the capture and elution of Staphylococcus aureus enterotoxin B (SEB) sample using silica tubing derivatized with an anti-SEB antibody.

### DETAILED DESCRIPTION OF THE INVENTION

In describing preferred embodiments of the present invention, specific terminology is employed for the sake of clarity. However, the invention is not intended to be limited to the specific terminology so selected. It is to be understood that each specific element includes all technical equivalents which operate in a similar manner to accomplish a similar purpose.

One aspect of the present invention relates to a simple and versatile fluidic device for purification and concentration of an analyte in a fluid sample. The invention utilizes a novel technique that generates a high surface area for capturing the analyte in a large volume sample, but allows low liquid elution volume for concentrating the analyte into a small volume. In principle, large boluses of sample are passed through a channel to deposit the analyte along the channel wall which either has a nature tendency, or is modified physically, chemically, or biologically, to bind to the analyte. Small boluses of fluid are then passed through the channel to elute and concentrate the analyte. As used herein, the term “channel” refers to any passages for fluid or liquid. A channel can be of any size and shape, such as a tubing, a groove on a surface, or a tubular passage in a substrate.

For example, the surface area of a 2 mm (id)×3 cm glass tubing is 188 mm². The relatively large diameter and liquid volume could easily accommodate large dirty samples. As the sample moves through the tubing, the analyte is deposited along the length of the tubing. Concentration of the analyte is accomplished by moving a small liquid bolus (e.g. 50 μl or 4 mm) of elution through the tubing, essentially collecting the bound analyte along the length of the tubing. Longer lengths of tubing could increase binding capacity, yet elution volumes would remain unchanged.

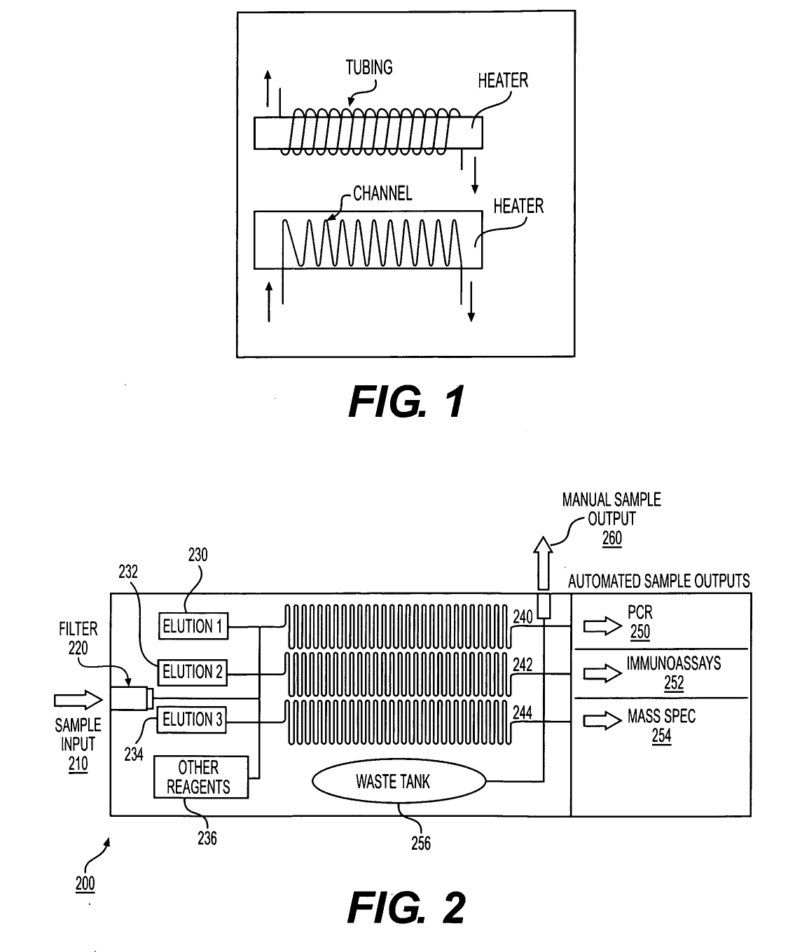

The relatively large diameter of the tubing would greatly facilitate tolerance to large particulates in the sample. However, laminar flow and tubular pinch, in which particles may not make contact with the tubing wall or move away from the tubing wall, could theoretically result in lower yields. These potential issues can be alleviated by using coiled tubing, which would create turbulence within the fluid flow and dramatically increase the likelihood of analyte contact with the tubing wall. Coiled tubing would also maintain a small footprint for the device. In addition, square or rectangular tubing can be used to minimize any tubular pinch effects, in which particles focus away from the walls. As shown in FIG. 1, coiled tubing could be wrapped around a heater, essentially resembling a solenoid, for enhanced nucleic acid elution. Alternatively, coiled tubing or channels can be placed, or constructed within a heating block. Heating the input sample would induce convection mixing within the tubing and increase kinetics of analyte binding to the tubing wall. Typically, the tubing or channel is connected to a fluid handling device, such as a pump with a multi-port valve, that introduces the sample fluid, washing buffer, elution buffer, and/or other reagents into the tubing or channel. Flow rates, diameters, lengths, temperatures, tubing material, curvature, etc. would be optimized initially with modeling then tested experimentally.

The channel-based device of the present invention occupies a relatively small footprint and can be designed to accommodate different analytical platforms. One skilled in the art would recognize that the device may comprise an array of individually functionalized channels. FIG. 2 shows an embodiment of a channel-based device 200 designed for three orthogonal downstream analytical platforms: polymerase chain reaction (PCR) 250, immunoassays 252, and mass spectrometry (MS) 254. A fluid sample is introduced into the device through a sample input 210, optionally passing through a filter 220 and mixed with reagents 236, and enters channels 240, 242 and 244. Each channel is designed to collect an analyte of interest. For example, channel 240 may be designed to collect polynucleotides for downstream analysis in the PCR platform 250; channel 242 may be designed to collect a protein or proteins for downstream analysis in the immunoassays platform 252; and channel 244 may be designed to collect a small molecule inhibitor for downstream analysis in the MS platform 250. The flow through of each channel is stored in waste tank 256, and discarded or recycled through a sample output 260. The analyte of interest in each channel is then eluted with a corresponding elution buffer 230, 232, or 234, and sent to the appropriate platform for further analysis. In one embodiment, each channel performs a specialized sample preparation protocol tailored to a specific analytical platform. The channel-based device of the present invention can be integrated in a complete analytical platform or used as a stand-alone sample preparation device.

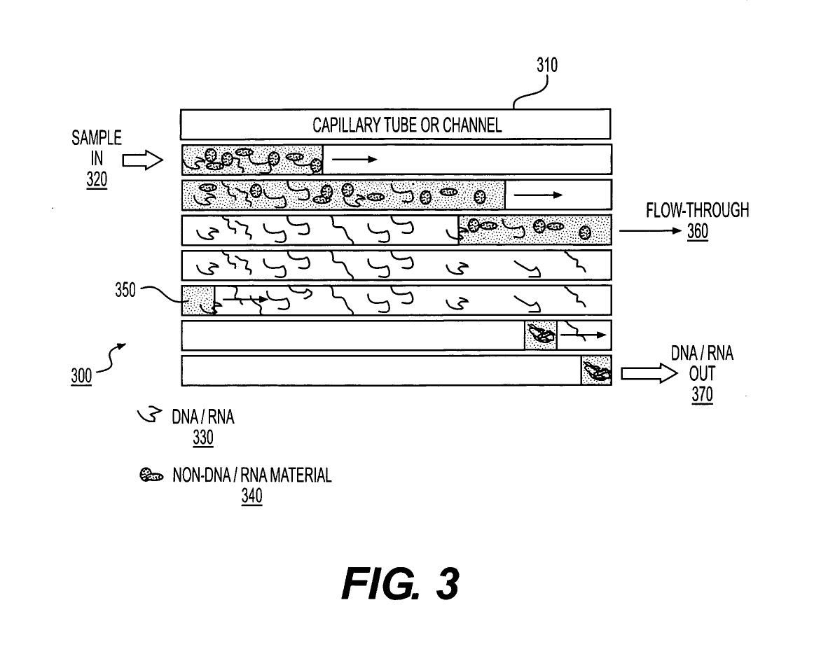

FIG. 3 depicts the working mechanism of a channel-based device 300 for nucleic acid purification. The device 300, designated as the microchannel nucleic acid concentrator (MNAC), uses a capillary tube or channel 310 to generate a high surface area, low liquid volume chamber for nucleic acid retention. When a sample 320 (e.g. 0.1-100 ml) mixed with guanidine (e.g. GuHCl, GuSCN) moves through the channel 310, nucleic acids 330 are deposited along the length of the channel wall. Non-nucleic acid materials 340, such as proteins and lipids, would stay in the flow-through 360. Unlike other approaches, concentration of the nucleic acids 330 is accomplished by moving a small liquid bolus (e.g. 5-100 μl) of elution buffer 350 through the channel 310, essentially collecting the bound nucleic acids 330 off the wall along the length of the channel 310 and exiting the channel 310 as eluant 370. The longer the channel length, the higher the binding capacity, yet elution volumes can remain unchanged. In one embodiment, the bolus of elution buffer is flanked by air. Compared to other nucleic acid purification devices, the MNAC has a low complexity and possesses several advantages as indicated in Table I.

TABLE I
Relative comparison of nucleic acid purification devices
with the current potential MNAC capabilities

| | | | Glass | Micro- | Para- | |
|---|---|---|---|---|---|---|
| | Qiagen | | bead | pillar | magnetic | |
| | membrane | Silica gel | column | chip | beads | MNAC |
| Very high | − | − | − | + | + | + |
| concentration | | | | | | |
| Inexpensive | + | + | +/− | − | + | + |
| Speed | − | − | − | + | + | + |
| Low variability | + | + | +/− | − | + | + |
| Tolerates large | − | − | − | − | + | + |
| particles | | | | | | |
| Binding capacity | + | + | + | − | + | + |
| Closed system | − | + | + | + | − | + |
| Simple automation/ | − | + | + | + | − | + |
| integration/packaging | | | | | | |
| Reusability | − | − | − | + | +/− | + |

The channel-based purification device of the present invention can be designed to capture a variety of analytes, ranging from biomolecules such as polypeptides, polynucleotides, polysaccharides, and lipids, to cells and virus particles.

In one embodiment, the analyte is genomic DNA from cells of interest. Examples of the cells of interest include, but are not limited to, eukaryotic and prokaryotic cells, parasites, bacteria, and virus particles. Examples of eukaryotic cells include all types of animal cells, such as mammal cells, reptile cells, amphibian cells, and avian cells, blood cells, hepatic cells, kidney cells, skin cells, brain cells, bone cells, nerve cells, immune cells, lymphatic cells, brain cells, plant cells, and fungal cells. In another aspect, the cells can be a component of a cell including, but not limited to, the nucleus, the nuclear membrane, leucoplasts, the microtrabecular lattice, endoplasmic reticulum, ribosomes, chromosomes, cell membrane, mitochondrion, nucleoli, lysosomes, the Golgi bodies, peroxisomes, or chloroplasts.

Examples of bacteria include, but are not limited to, Abiotrophia, Achromobacter, Acidaminococcus, Acidovorax, Acinetobacter, Actinobacillus, Actinobaculum, Actinomadura, Actinomyces, Aerococcus, Aeromonas, Afipia, Agrobacterium, Alcaligenes, Alloiococcus, Alteromonas, Amycolata, Amycolatopsis, Anaerobospirillum, Anaerorhabdus, Arachnia, Arcanobacterium, Arcobacter, Arthrobacter, Atopobium, Aureobacterium, Bacteroides, Balneatrix, Bartonella, Bergeyella, Bifidobacterium, Bilophila Branhamella, Borrelia, Bordetella, Brachyspira, Brevibacillus, Brevibacterium, Brevundimonas, Brucella, Burkholderia, Buttiauxella, Butyrivibrio, Calymmatobacterium, Campylobacter, Capnocytophaga, Cardiobacterium, Catonella, Cedecea, Cellulomonas, Centipeda, Chlamydia, Chlamydophila, Chromobacterium, Chyseobacterium, Chryseomonas, Citrobacter, Clostridium, Collinsella, Comamonas, Corynebacterium, Coxiella, Cryptobacterium, Delftia, Dermabacter, Dermatophilus, Desulfomonas, Desulfovibrio, Dialister, Dichelobacter, Dolosicoccus, Dolosigranulum, Edwardsiella, Eggerthella, Ehrlichia, Eikenella, Empedobacter, Enterobacter, Enterococcus, Erwinia, Erysipelothrix, Escherichia, Eubacterium, Ewingella, Exiguobacterium, Facklamia, Filifactor, Flavimonas, Flavobacterium, Francisella, Fusobacterium, Gardnerella, Gemella, Globicatella, Gordona, Haemophilus, Hafnia, Helicobacter, Helococcus, Holdemania Ignavigranum, Johnsonella, Kingella, Klebsiella, Kocuria, Koserella, Kurthia, Kytococcus, Lactobacillus, Lactococcus, Lautropia, Leclercia, Legionella, Leminorella, Leptospira, Leptotrichia, Leuconostoc, Listeria, Listonella, Megasphaera, Methylobacterium, Microbacterium, Micrococcus, Mitsuokella, Mobiluncus, Moellerella, Moraxella, Morganella, Mycobacterium, Mycoplasma, Myroides, Neisseria, Nocardia, Nocardiopsis, Ochrobactrum, Oeskovia, Oligella, Orientia, Paenibacillus, Pantoea, Parachlamydia, Pasteurella, Pediococcus, Peptococcus, Peptostreptococcus, Photobacterium, Photorhabdus, Plesiomonas, Porphyrimonas, Prevotella, Propionibacterium, Proteus, Providencia, Pseudomonas, Pseudonocardia, Pseudoramibacter, Psychrobacter, Rahnella, Ralstonia, Rhodococcus, Rickettsia Rochalimaea Roseomonas, Rothia, Ruminococcus, Salmonella, Selenomonas, Serpulina, Serratia, Shewenella, Shigella, Simkania, Slackia, Sphingobacterium, Sphingomonas, Spirillum, Staphylococcus, Stenotrophomonas, Stomatococcus, Streptobacillus, Streptococcus, Streptomyces, Succinivibrio, Sutterella, Suttonella, Tatumella, Tissierella, Trabulsiella, Treponema, Tropheryma, Tsakamurella, Turicella, Ureaplasma, Vagococcus, Veillonella, Vibrio, Weeksella, Wolinella, Xanthomonas, Xenorhabdus, Yersinia, and Yokenella. Other examples of bacterium include Mycobacterium tuberculosis, M. bovis, M. typhimurium, M. bovis strain BCG, BCG substrains, M. avium, M. intracellulare, M. africanum, M. kansasii, M. marinum, M. ulcerans, M. avium subspecies paratuberculosis, Staphylococcus aureus, Staphylococcus epidermidis, Staphylococcus equi, Streptococcus pyogenes, Streptococcus agalactiae, Listeria monocytogenes, Listeria ivanovii, Bacillus anthracis, B. subtilis, Nocardia asteroides, and other Nocardia species, Streptococcus viridans group, Peptococcus species, Peptostreptococcus species, Actinomyces israelii and other Actinomyces species, and Propionibacterium acnes, Clostridium tetani, Clostridium botulinum, other Clostridium species, Pseudomonas aeruginosa, other Pseudomonas species, Campylobacter species, Vibrio cholerae, Ehrlichia species, Actinobacillus pleuropneumoniae, Pasteurella haemolytica, Pasteurella multocida, other Pasteurella species, Legionella pneumophila, other Legionella species, Salmonella typhi, other Salmonella species, Shigella species Brucella abortus, other Brucella species, Chlamydi trachomatis, Chlamydia psittaci, Coxiella burnetti, Escherichia coli, Neiserria meningitidis, Neiserria gonorrhea, Haemophilus influenzae, Haemophilus ducreyi, other Hemophilus species, Yersinia pestis, Yersinia enterolitica, other Yersinia species, Escherichia coli, E. hirae and other Escherichia species, as well as other Enterobacteria, Brucella abortus and other Brucella species, Burkholderia cepacia, Burkholderia pseudomallei, Francisella tularensis, Bacteroides fragilis, Fudobascterium nucleatum, Provetella species, and Cowdria ruminantium, or any strain or variant thereof.

Examples of viruses include, but are not limited to, Herpes simplex virus type-1, Herpes simplex virus type-2, Cytomegalovirus, Epstein-Barr virus, Varicella-zoster virus, Human herpesvirus 6, Human herpesvirus 7, Human herpesvirus 8, Variola virus, Vesicular stomatitis virus, Hepatitis A virus, Hepatitis B virus, Hepatitis C virus, Hepatitis D virus, Hepatitis E virus, Rhinovirus, Coronavirus, Influenza virus A, Influenza virus B, Measles virus, Polyomavirus, Human Papilomavirus, Respiratory syncytial virus, Adenovirus, Coxsackie virus, Dengue virus, Mumps virus, Poliovirus, Rabies virus, Rous sarcoma virus, Yellow fever virus, Ebola virus, Marburg virus, Lassa fever virus, Eastern Equine Encephalitis virus, Japanese Encephalitis virus, St. Louis Encephalitis virus, Murray Valley fever virus, West Nile virus, Rift Valley fever virus, Rotavirus A, Rotavirus B, Rotavirus C, Sindbis virus, Simian Immunodeficiency cirus, Human T-cell Leukemia virus type-I, Hantavirus, Rubella virus, Simian Immunodeficiency virus, Human Immunodeficiency virus type-1, Vaccinia virus, SARS virus, and Human Immunodeficiency virus type-2, or any strain or variant thereof.

Examples of parasites include, but are not limited to, Toxoplasma gondii, Plasmodium falciparum, Plasmodium vivax, Plasmodium malariae, other Plasmodium species, Trypanosoma brucei, Trypanosoma cruzi, Leishmania major, other Leishmania species, Schistosoma mansoni, other Schistosoma species, and Entamoeba histolytica, or any strain or variant thereof.

Channels of the purification device can be made of glass, plastic, ceramic, silicon, silicone or any other suitable materials. The interior of the channel either has a nature tendency, or is modified physically, chemically, or biologically, to bind to the analyte of interest when the sample fluid passes through the channel. In one embodiment, the analyte is genomic DNA and the channels are either glass or fused silica tubing. In another embodiment, the analyte is a polypeptide, the channel wall is derivertized with an antibody to capture the analyte.

Serpentine channels are preferred over linear channels, since longer channels can be compacted into a smaller space. In addition, laminar flow, in which particles may not make contact with the channel wall would be alleviated due to the mixing effects of the channel curves. The channel walls may be modified to increase turbulence.

The diameter of the channels is selected based on the intended use of the device. Generally speaking, large diameter of the channel (e.g., >200 um) facilitates tolerance to large particulates in the sample. As shown in FIG. 2, a frit or filter 220 may be placed upstream of the inlet of the channels to prevent large particles from blocking the inlet. Frit pore size would be slightly smaller than channel diameter. The channels may have gradient diameters (e.g., starts off as a very large diameter and transitions to a smaller diameter). Gradient channels can facilitate an even distribution of the analytes along the length of the channel wall, which will prevent loading up of analytes on the channel wall near the inlet. Additional equipments, such as a bead-beater or sonicator, may also be employed to pretreat the sample before it enters the channels.

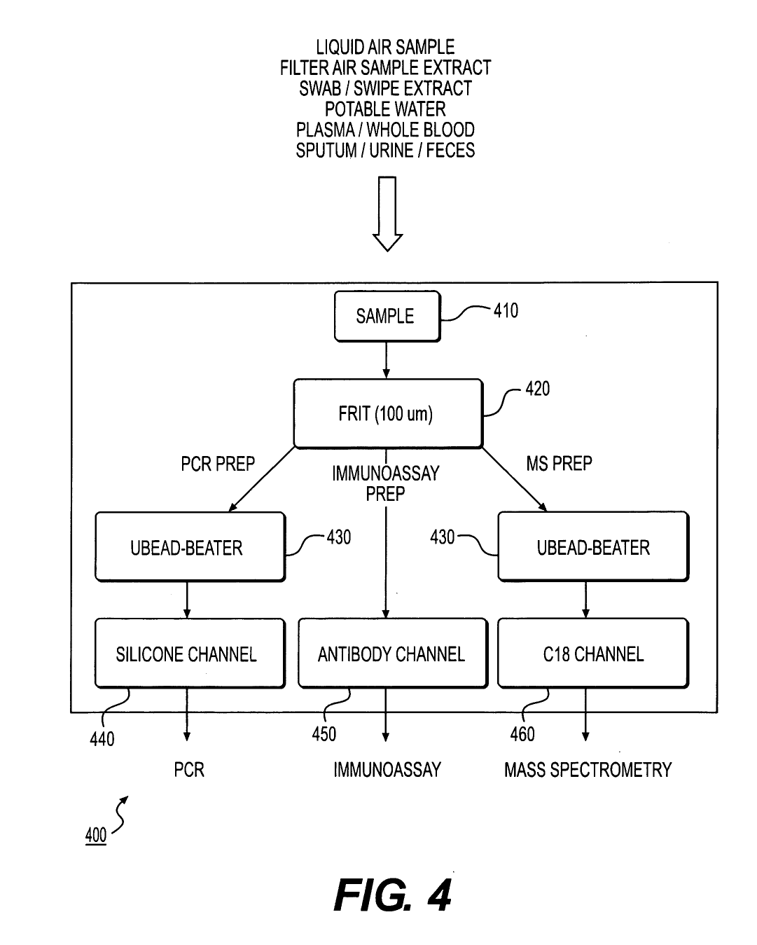

FIG. 4 shows an embodiment of a channel-based approach to sample preparation for PCR, immunoassays, and MS. Briefly, sample 410 is filtered through a frit 420 and then divided into three streams for subsequent analyte collection in silicone channel 440, antibody channel 450, and C18 channel 460. In one embodiment, filtered samples 410 are pretreated with ubead-beater 430 before entering the channels 440, 450 and 460. In another embodiment, components of the channel-based device 400 are modular to allow different approaches and configurations.

Stringency of the binding and elution of the analyte to the channels can be controlled by binding and elution buffer formulations. For example, elution stringencies in anion exchange columns for proteins and nucleic acids can be controlled by salt concentrations using KCl or NaCl. Nucleic acids, with their higher negative charge, are more resistant to elution than proteins. Temperature, pH, and mild detergent are other treatments that could be used for selective binding and elution. In one embodiment, the sample fluid is preheated to a temperature of 45° C. to 85° C., before entering the channels. In another embodiment, the analyte of interest is eluted from the channel by filling the channel with an elution buffer, incubating the filled channel at an elevated temperature (e.g., 45° C. to 85° C.) for a desired period of time (e.g., 2-30 minutes, preferably 5-15 minutes), and then releasing the elution buffer from the capillary channel. Thermal consistency of the binding and elution may be maintained with a heat block or a water bath.

The inner surface of the channel may be derivatized with chemistries to functionalize the surfaces, particularly for plastics. For example, channels may be derivatized in polymethylmethacrylate (PMMA) and cyclo-olefin-copolymer COC with antibodies as capture moieties for toxin. Other candidate materials for surface functionalization include lectins (binds carbohydrates found in bacteria coats), aminosilanes (creates positive charge), and charge-switch technology. Other capture methods, such as lectins on membranes (Bundy and Fenselau, Anal Chem 1999; 71:1460-3) and antibodies on magnetic beads (Madonna et. al., Rapid Commun Mass Spectrom 2001; 14:2220-9), can be readily implemented into the channel-based format. The channel based device may also contain a pump for sample/reagent delivery and/or a microprocessor that controls the binding/washing/eluting procedures.

Another aspect of the present invention relates to a method for purifying an analyte of interest from a fluid sample. The method comprises the steps of: passing the fluid through a capillary channel having an inner surface that binds to the analyte of interest; washing the capillary channel with a washing buffer; and eluting the bound analyte with a elution buffer. In one embodiment, the method further comprises: pretreating the fluid sample before the passing step. In another embodiment, the eluting step comprises: filling the capillary channel with the elution buffer, incubating the filled channel at an elevated temperature, and releasing the elution buffer from the capillary channel. In another embodiment, the analyte is a polynucleotide. In another embodiment, the analyte is a protein. In yet another embodiment, the inner surface of the capillary channel is derivatized with an antibody.

### EXAMPLES

### Example 1

### Purification of DNA Using Capillary Tubing and Channels

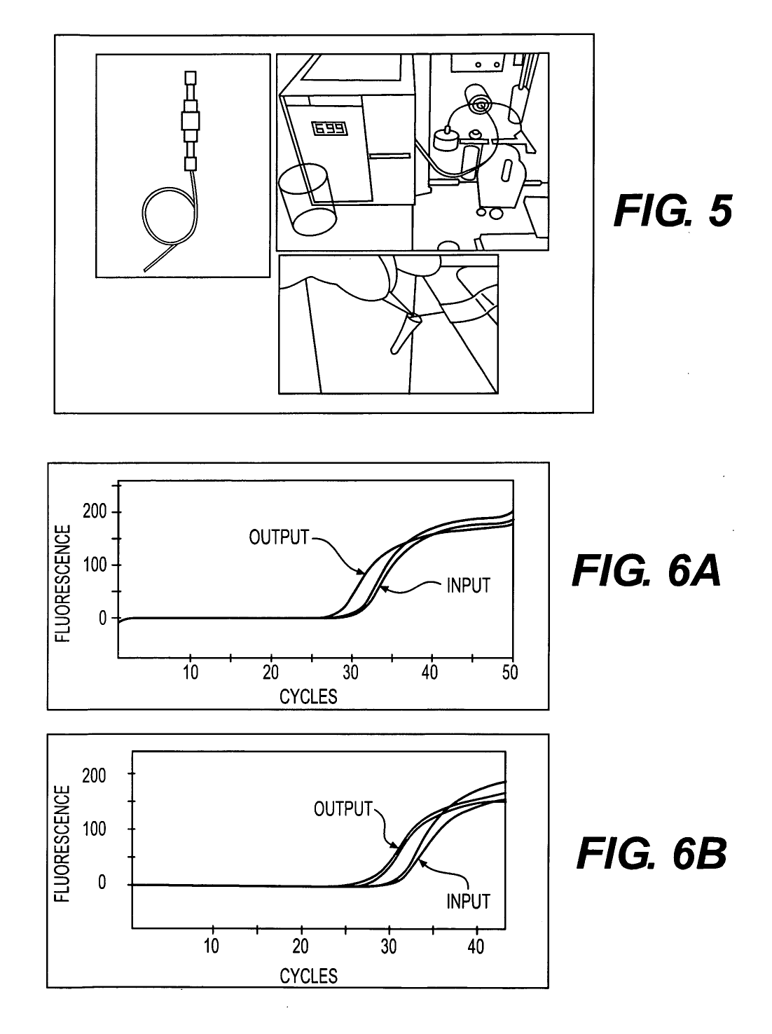

FIG. 5 shows a testbed using a coiled silica capillary (top left panel). A sample/guanidine mixture was loaded into the capillary by a syringe drive and multi-port valve (top right panel). The sample mixture was then moved through the capillary by a Global FIA pump to deposit DNA along the capillary wall. The capillary was washed first with ethanol to remove any residual proteins that also became associated with the capillary wall, followed with air to remove trace ethanol. Next, a small bolus of elution solution was moved through the capillary to elute the DNA off the capillary wall and deposit the concentrated DNA into a collection vial (bottom panel). The eluted DNA was quantitated by real time PCR (TaqMan) analysis using a standard curve. After each test, the capillary was decontaminated with 10% bleach.

After substantial testing of various configurations for MNAC, a protocol was established for early performance evaluation. This protocol processed a 300 μl input sample of 10⁵ copies M13 DNA (3,333 copies/μl) in 1:1 GuHCl, pH 6.8, using a 200 μm id×50 in fused silica capillary. The binding conditions were 5 min with continuous flow. Bound DNA was eluted with 30 μl 0.01N NaOH, pH12, with a 15 min hold at 75° C. PCR results in FIG. 6A demonstrate successful MNAC performance. The input M13 DNA was concentrated by nearly 10-fold, with the median recovery of the M13 DNA in 4 runs being 75.3%. The experiment was repeated with a fast elution time, i.e., 5 min hold at 75° C. The mean recovery for 4 runs was 41.86% (FIG. 6B).

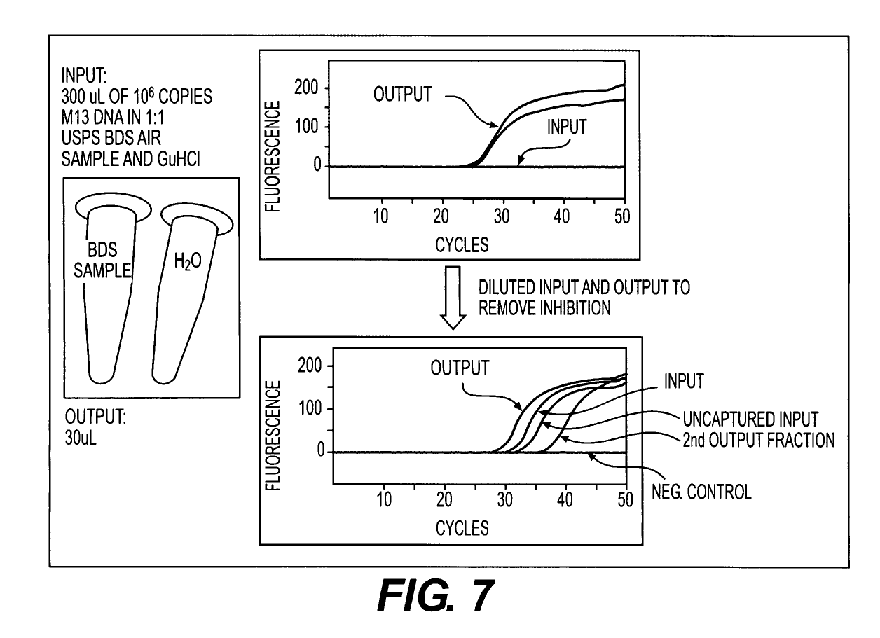

To assess the effects of a “dirty” sample on recovery, M13 DNA was spiked into air sampler fluid. This sample had been collected by a Northrop Grumman Biohazard Detection System (BDS sample) deployed at a United States Postal Service (USPS) mail sorting facility. The top panel of FIG. 7 shows the very inhibitory effects of the BDS sample (not processed by the on-board GeneXpert®) on PCR. MNAC processing of this sample produced a positive PCR signal. To demonstrate that the output sample was indeed concentrated, the input, the output (first 30 μl fraction), second output (second 30-μl fraction), and the input sample after passing through the MNAC (represents uncaptured DNA in the guanidine mixture), were diluted to remove the inhibitory effects of the air sample (input and uncaptured input) and the guanidine (uncaptured input). The results in the bottom panel show that the output represented concentrated M13 DNA and 88.6% of the DNA was recovered in the first 30-μl output fraction.

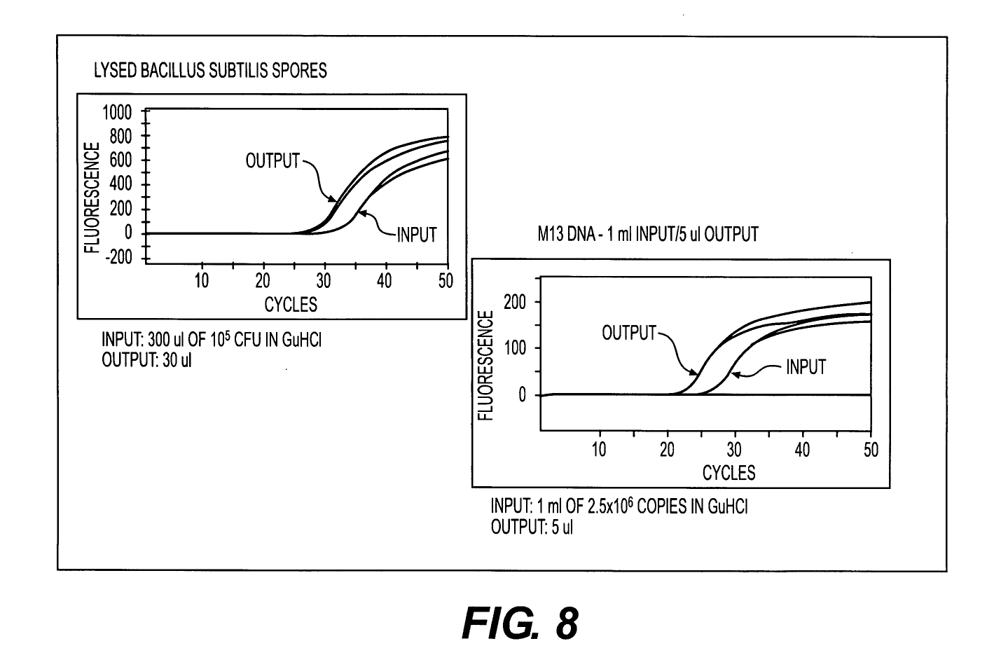

FIG. 8 shows results from other feasibility studies. The MNAC can be used to effectively capture DNA from lysed Bacillus subtilis spores (left panel) and to concentrate M13 DNA from a 1-ml input into a 5-μl output (right panel).

### Example 2

### Alternative Materials and Chemistries

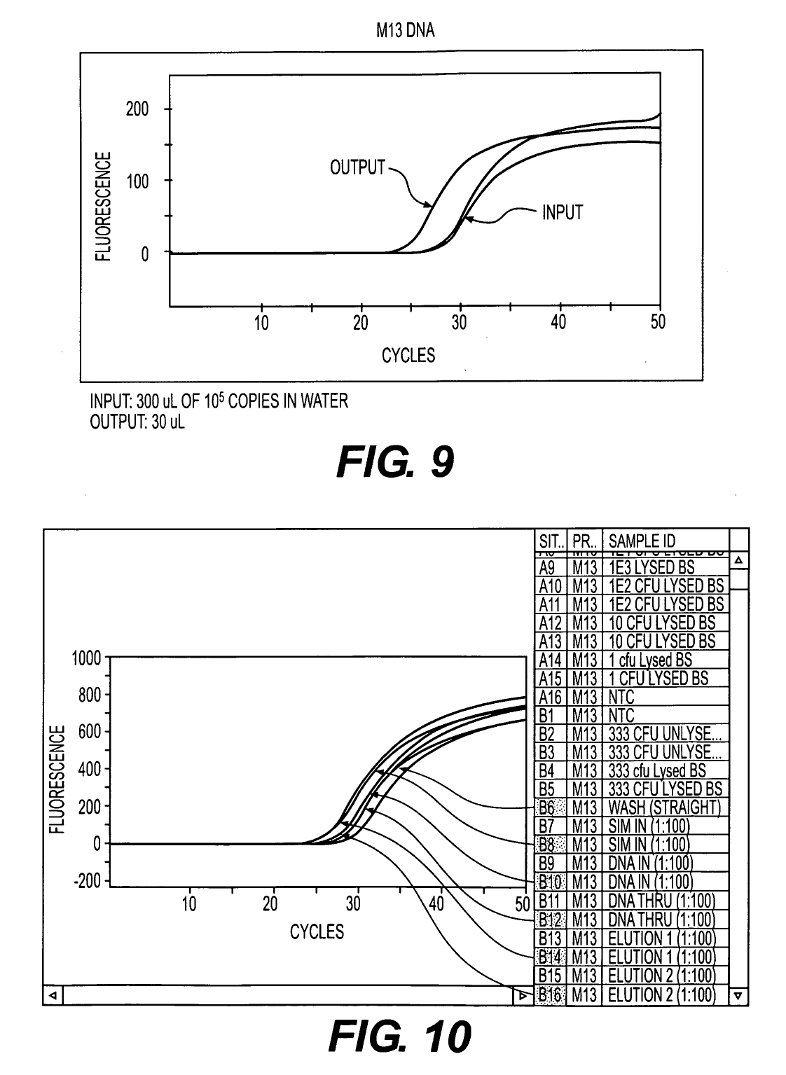

Materials other than glass were screened to extract and elute nucleic acids. Precedence for this is based on experiences with microfluidics in which the undesired, but not well-defined, effects of DNA loss to certain materials are observed. Thus, if these nucleic acid affinity properties can be exploited and optimized, new and simpler approaches to purify nucleic acids and other analytes can be developed. FIG. 9 shows concentration results using silicone tubing and a “clean” sample of M13 DNA (without guanidine and ethanol). The median recovery for 6 tests was 71.1%.

FIG. 10 shows the results of purification of M13 DNA with silicone tubing and a modified elution buffer. Briefly, 1×10⁶ copies of M13 DNA was suspended in 300 μl 75% ChargeSwitch® binding buffer (Invitrogen, Carlsbad, Calif.). The DNA suspension was loaded into the silicone tubing at a flow rate of 0.28 μl/sec (total loading time 15 min). The tubing was washed with 120 μl ChargeSwitch® washing buffer (Invitrogen, Carlsbad, Calif.) at a flow rate of 3 μl/sec. The DNA was eluted with 30 μl elution buffer (1×PBS with 0.01% SDS, pH9.0) at 75° C. for 15 min in flow-hold script mode (i.e. moving the elution buffer, stopping the elution buffer and holding, moving the elution buffer again, etc). The temperature was maintained by a water bath. The recovery rate was 30.3%.

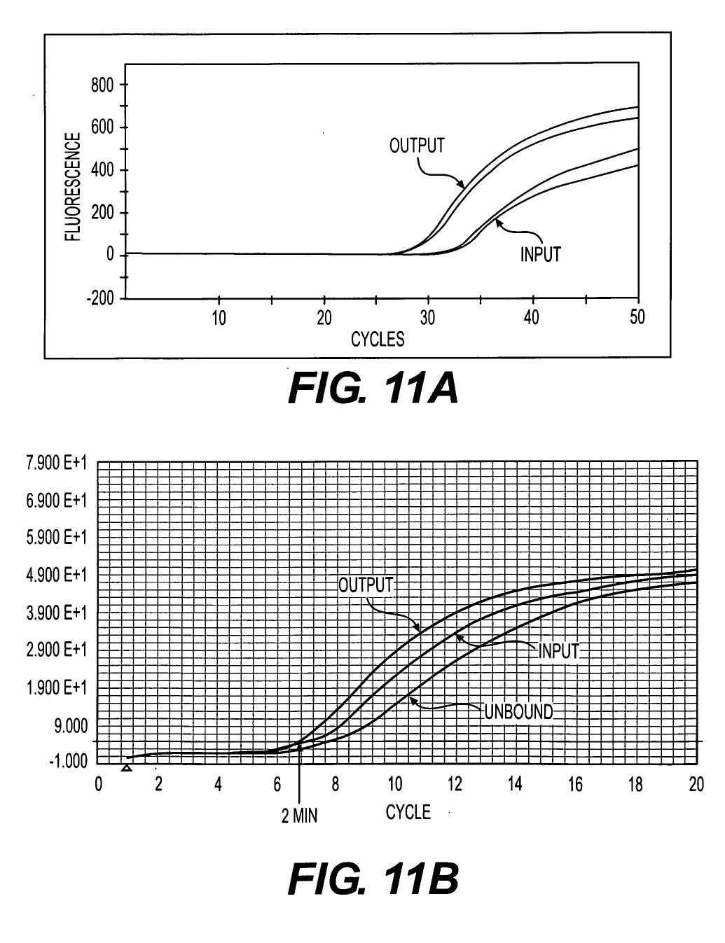

FIGS. 11A and 11B show the results of channel-based purification of DNA from lysed Bacillus subtilis (BS) spores. 300 μl of lysed BS spores (333 cfu/μl in 1:1 GuHCl) was loaded into the channels for 5 min with continuous flow. The bound DNA was eluted with 30 μl 0.01N NaOH, pH12, with a 5 min hold at 75° C. The original sample (input), eluted sample (output), and flow-through sample (unbound) were analyzed by real-time PCR (FIG. 11A) and real-time isothermal amplification (FIG. 11B). The results showed that the channel-based purification method of the present invention can be used to concentrate DNA from lysed BS spores in a test sample.

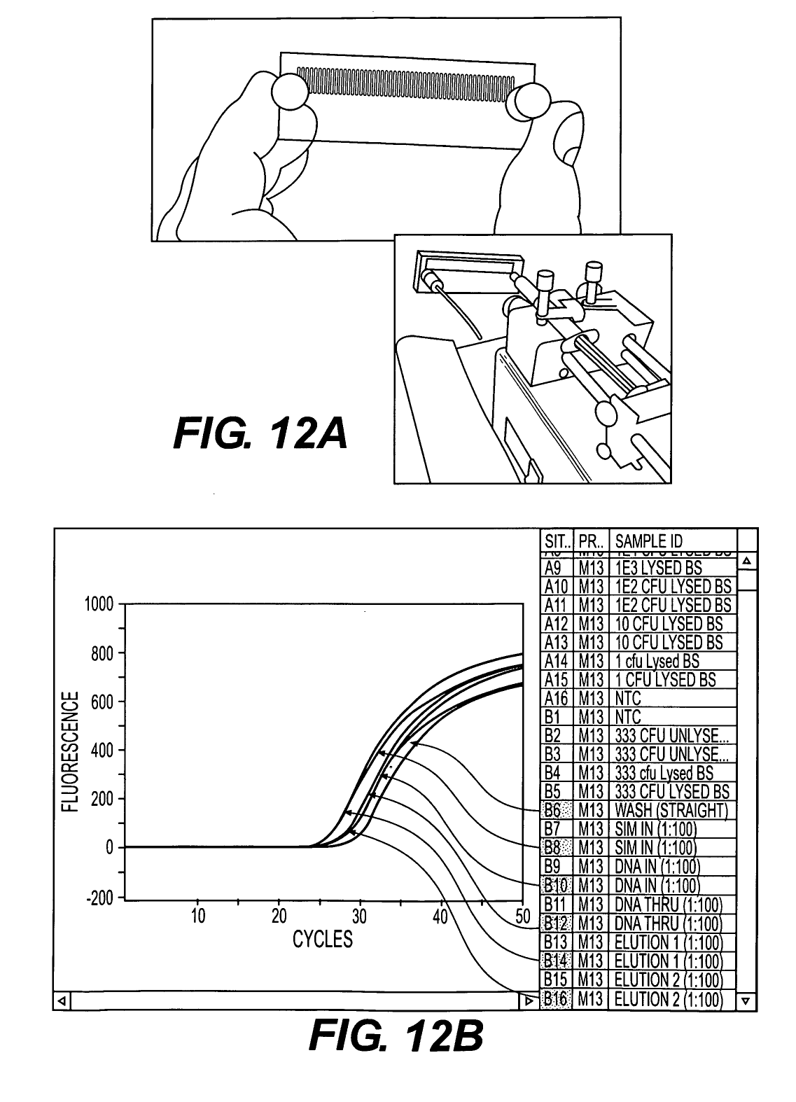

FIG. 12A shows an aminated microfluidic serpentine channel in a PMMA chip (upper panel) and a syringe pump to move fluids through the chip (lower panel). FIG. 12B shows the results of purification of DNA from lysed Bacillus anthracis spores with aminated PMMA microfluidic channel. Briefly, 1×10⁵ copies of lysed Bacillus anthracis spores was suspended in 300 μl 75% ChargeSwitch® binging buffer. The DNA suspension was loaded into the PMMA microfluidic channel at a flow rate of 0.34 μl/sec (total loading time 15 min). The tubing was washed with 300 μl ChargeSwitch® washing buffer at a flow rate of 3 μl/sec. The DNA was eluted with 30 μl elution buffer (10 mM NaOH, pH12.0) in a period of 15 min. The elution conditions were forward flow at 0.17 μl/sec for 50 μl, holding for 5 min and forward flow again. The elution temperature was 75° C. maintained by a heat block. The recovery rate was 21.7%.

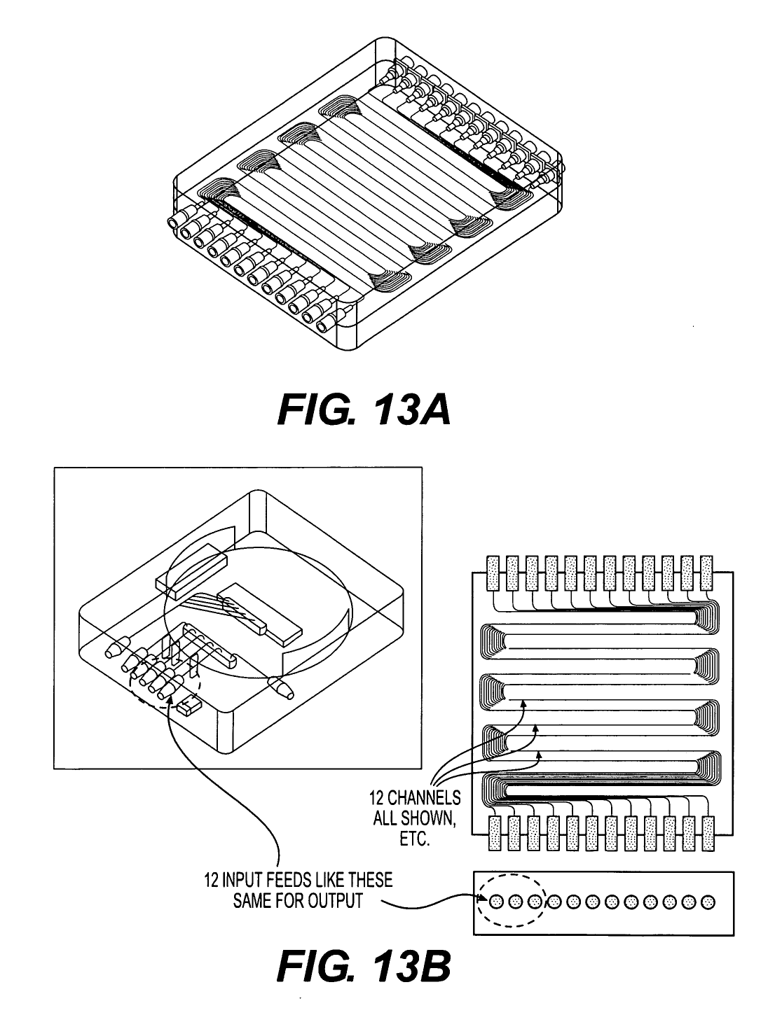

Multiple channels may be assembled together to simultaneously process multiple samples. FIGS. 13A and 13B show embodiments of channel modules that comprise multiple parallel channels that can process many samples at the same time.

### Example 3

### Sample Preparation for PCR Protocol Development

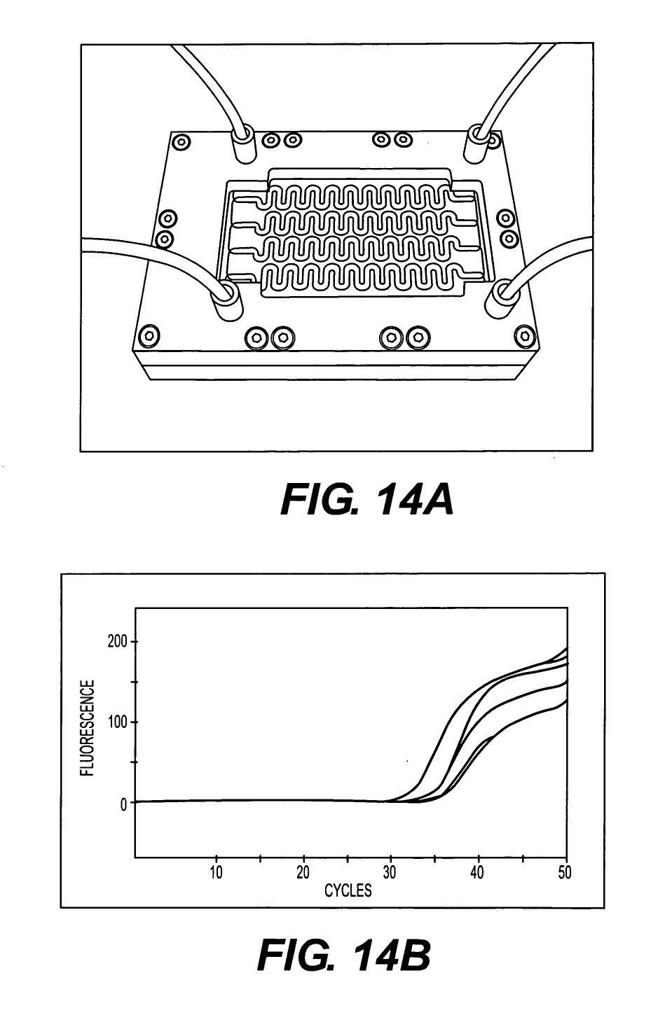

A commercially available silica serpentine channel (FIG. 14A) from Invenios (Santa Barbara, Calif.) was tested. The channel liquid volume was 1 ml. 10 ml M13 DNA at 2000 copies/μl in 1:1 GuHCl was loaded for 5 min with continuous flow. The bound DNA was eluted with 170 μl of 0.01N NaOH, pH12, with a 15 min hold at 75° C. FIG. 14B shows the concentrating effect of the serpentine channel. For Bacillus genomic DNA/spores and MS2 RNA/virions, a front-end lysis component, such as flow-through μBead-beater could be implemented upstream. Target concentrations ranging from 1-10⁶ copies are subjected to processing. Concentrated nucleic acids are quantitated by real-time PCR. Recoveries on the channel-based device are compared to that obtained using Qiagen kits.

For RNA, RNase inhibitors may be required. When cells are lysed, RNases can be released that degrade target RNA. For the chaotrope/silica method, guanidine will inhibit RNases. For non-guanidine approaches, RNase inhibitors can be introduced prior to or following lysis. Such RNase inhibors include RNAsin® (Becton-Dickinson, Franklin Lakes, N.J.), SUPERase-In™ (Ambion, Austin, Tex.), and ScriptGuard™ (Cambio Ltd, Cambridge, UK). Since most RNase inhibitors are proteins that bind to the RNase, it is feasible and cost-effective to coat a channel with the RNase inhibitor to extract the RNases from the sample. This concept of using channels to deplete the sample of an interferent by extracting the interferent instead of the target analyte is another use of channels for reducing sample complexity.

### Example 4

### Sample Preparation for Immunoassay Protocol Development

For immunoassays, glass or plastic channels are derivatized with antibodies for the analyte of interest. Elution will be performed using a low pH buffer. The elution buffer is neutralized for subsequent immunoassay testing using sandwich assays and/or lateral flow strips. Target concentrations ranging from 10 pg-100 ng are subjected to processing. Captured target analytes are eluted and concentrated, then subjected to downstream detection and identification. To utilize the channel-based device for immunoassay, one or more channels are derivatized with an antibody of a mixture of antibodies for the analyte(s) of interest. Antibodies that exhibit higher cross-reactivity, lower specificity could actually be more useful for this sample processing approach. Since the capture antibodies are immobilized, a mixed population of these antibodies should not interfere with one another in the extraction/binding/elution process. Highly specific antibodies could be used for downstream detection and identification.

In one experiment, clean M13 DNA sample was purified using a silica tubing derivatized with anti-DNA antibodies. The preliminary results suggest that the capture efficiency was about 50%.

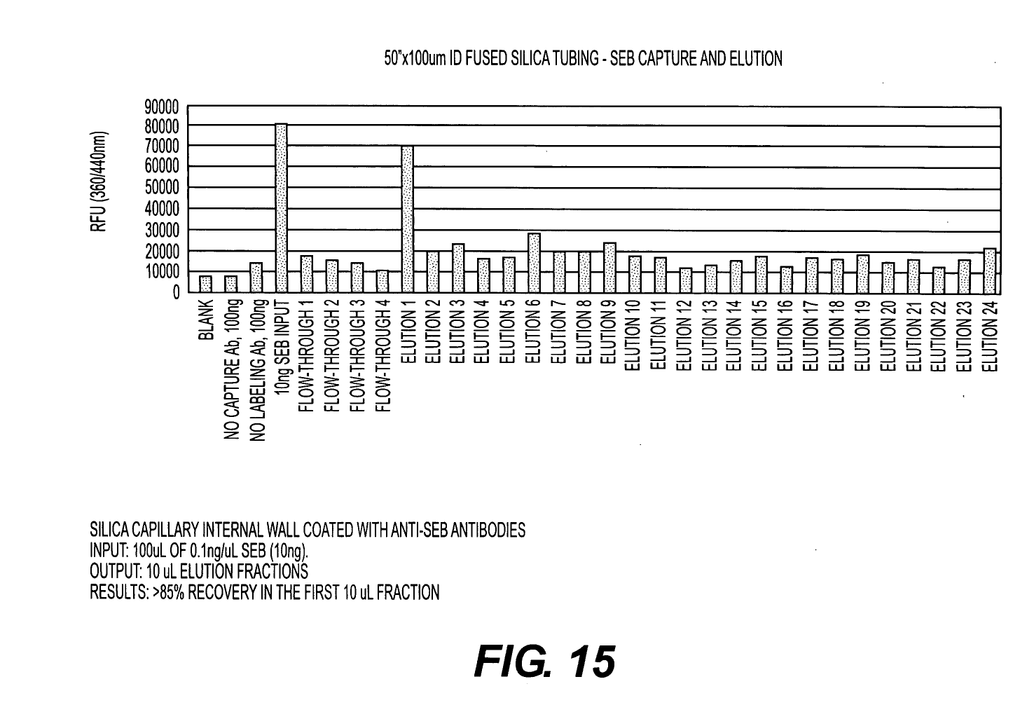

In another experiment, clean Staphylococcus aureus, enterotoxin B (SEB) sample was purified using silica tubing derivatized with an anti-SEB antibody (input: 100 μl of SEB at 0.1 ng/ul; output: 10 μl elution fractions). As shown in FIG. 15, the recovery rate is about 85% in the first 10 μl elution fraction.

### Example 5

### Sample Preparation for MS Protocol Development

Mass spectrometry (MS) has emerged as a powerful diagnostic tool for the differentiation and identification of cultured bacteria and remains a promising approach for the identification of bacteria in clinical or environmental samples. In these types of samples, microbial and toxin targets can be present at relatively low levels in a complex background containing salts, debris, and other contaminants which are known to have deleterious effects on the MS signal. To obtain reproducible, reliable MS signal from these samples it is critical to separate and concentrate the targets from the background and remove any components that may cause MS sample suppression.

Bacteria have a number of surface characteristics which should allow for selective concentration of the organism from complex matrices. These include the presence of surface-exposed carbohydrates, a net-negative charge at high pHs, and protein antigens. Some of these characteristics have been exploited for MS separation and concentration including the use of lectins, carbohydrate-binding proteins of non-immune origin which bind carbohydrates, immunomagnetic beads which specifically bind antigens, and the use of carbohydrates which bind other surface-specific proteins. These protocols have almost always used small immobilization areas (as opposed to channels) which have required extensive washing steps to purify the sample for direct MS analysis of the surface. Additionally, after the bind and wash steps these protocols have required some type of chemical pretreatment of the bacteria to release proteins for analysis.

A channel-based device can be developed for performing a bacterial sample separation and concentration for MS analysis. Several purification chemistries can be used in the device, including but are not limited to, lectin and carbohydrate-based separations, hydroxyapatite (HAP), protein-based separations using lysozyme and bovine serum albumin (BSA), as well as hydrophobicity/charge based separation using polymer surfaces such as C4, C18, and polyethylene glycol (PEGs). The methods for immobilizing the above-mentioned moieties to glass and plastic surfaces are well known to one skilled in the art. For each of these chemistries the optimum binding and elution conditions, including the effect of pH, MS compatible salts and buffers, organic concentration, temperature, and the binding efficiency and concentration factor will be determined.

Cell disruption or lysis is recognized as being useful for effective MS analysis. This disruption can be accomplished by either chemical or physical means. In one embodiment, physical disruption of cells will be physically disrupted prior to MS cleanup using the μBead-beater. In this case, the target compounds become proteins instead of whole cells. For lysed target, sample concentration and separation may be more effective using immobilized polymer phases (C4, C18) which generically bind most of the protein species in aqueous solution. Protein antigen and carbohydrate specific chemistries, while useful for whole bacteria capture, would be limited to more specific protein capture following lysis. This could still be useful, since instead of trying to analyze a complex protein signature, identification would be limited to a panel of discriminating proteins.

Another aspect of the present invention relates to a method for purifying an analyte of interest from a fluid sample. The method comprises the steps of: passing the fluid through a capillary channel having an inner surface that binds to the analyte of interest; washing the capillary channel with a washing buffer; and eluting the bound analyte with an elution buffer. In one embodiment, the method further comprises: pretreating the fluid sample before the passing step. In another embodiment, the eluting step comprises: filling the capillary channel with the elution buffer, incubating the filled channel at an elevated temperature, and releasing the elution buffer from the capillary channel. In another embodiment, the analyte is a polynucleotide. In another embodiment, the analyte is a protein. In yet another embodiment, the inner surface of the capillary channel is derivatized with an antibody.

The embodiments illustrated and discussed in this specification are intended only to teach those skilled in the art the best way known to the inventors to make and use the invention. Nothing in this specification should be considered as limiting the scope of the present invention. The above-described embodiments of the invention may be modified or varied, and elements added or omitted, without departing from the invention, as appreciated by those skilled in the art in light of the above teachings. It is therefore to be understood that, within the scope of the claims and their equivalents, the invention may be practiced otherwise than as specifically described.

What is claimed is:

1. A method for purifying an analyte from a fluid sample, comprising:
passing said fluid sample through a channel having an inner surface that binds to said analyte; and
eluting bound analyte with a elution buffer.

2. The method of claim 1, further comprising:
washing said channel with a washing buffer.

3. The method of claim 1, wherein said elution buffer has a volume that is ten times less than the volume of said fluid sample.

4. The method of claim 1, further comprising:
pretreating said fluid sample before the passing step.

5. The method of claim 1, wherein said eluting step comprises:
filling the channel with the elution buffer;
incubating the filled channel at an elevated temperature for a desired period of time; and
releasing the elution buffer from the channel.

6. The method of claim 5, wherein said elevated temperature is in the range of 45° C. to 85° C.

7. The method of claim 5, wherein said desired period of time is between 2 and 30 minutes.

8. The method of claim 1, wherein said channel is embedded in a microfluidic circuit.

9. The method of claim 1, wherein said analyte is a polynucleotide or a polypeptide.

10. The method of claim 1, wherein said channel is reusable for purification and concentration of sequential samples.

11. The method of claim 10, further comprising:
passing a decontamination solution through the channel after the analyte is eluted to prepare the channel for the next sample.

12. The method of claim 1, wherein the inner surface of the channel is derivatized with an antibody or lectins.

13. The method of claim 1, further comprising the step of:
passing the fluid sample through a frit or filter.

14. The method of claim 1, further comprising the step of:
pretreating said fluid sample with a bead-beater or sonicator.

15. A device for purifying an analyte from a fluid sample, comprising:
a channel having an inner surface that binds to said analyte in said fluid sample;
a fluid handling unit capable of injecting an elution buffer into said channel to elute analyte bound to the inner surface of said channel; and
a heating unit'capable of heating fluid in said channel for a desired period of time.

16. The device of claim 15, wherein said channel is a serpentine channel.

17. The device of claim 15, wherein said channel is made of glass, silicone or polymethylmethacrylate (PMMA).

18. The device of claim 15, further comprising multiple channels capable of simultaneously processing multiple fluid samples.

19. The device of claim 15, further comprising a filter placed up-stream of the channel.

20. The device of claim 15, further comprising a microprocessor that controls the fluid handling unit and the heating unit.
